(19) United States
(12) Patent Application Publication
(10) Pub. No.: US 2012/0022366 A1
(43) Pub. Date: Jan. 26, 2012

(54) REGISTRATION OF AORTA TO PATIENT VIA TWO 2D IMAGES FOR PLACEMENT OF A STENT

(76) Inventor: Marcus Pfister, Bubenreuth (DE)

(21) Appl. No.: 12/840,649

(22) Filed: Jul. 21, 2010

(51) Int. Cl. A61B 5/05

(52) U.S. Cl. 600/427

(57) ABSTRACT

In a method for visualizing placement of a stent in an aorta of a patient with reduced use of contrast agent, a 3D volume image of the aorta of the patient is provided from a CT scan before placing the stent. An angiography system with a C-arm is provided to take 2D images of the patient. A computer is provided having registration software for registering the 3D volume image and 2D images taken by the angiography system. A first segmentation is performed on the 3D volume image to segment the aorta from remaining parts of the 3D image. A second segmentation is performed using the first segmentation on the 3D volume image to segment a bony structure of the patient from remaining parts of the 3D volume image. A first 2D image of the aorta is obtained from a first direction with use of a contrast agent. A second 2D image is obtained from a second direction but without use of contrast agent. The segmented aorta in the 3D volume image is registered to the C-arm to create a registered 3D volume image by registering the first 2D image to the segmented aorta and registering the second 2D image to the segmented bony structure. The stent is placed in the aorta while observing on the angiography system a third continuous 2D image taken by the angiography system superimposed on the registered 3D volume image.

### BACKGROUND

This disclosure relates to fluoroscopy controlled, interventional repair of aortic aneurysms such as shown in prior art FIG. 1a, and particularly Abdominal Aortic Aneurysms 10 (AAA) which is a disease of the abdominal segmented aorta 9. This disease is usually treated by inserting stent grafts into the aorta to remodel the organ. Through the groins, guide wires 11 and catheters are inserted (prior art FIG. 1b) with which one or more stent grafts 12 (also called “stents” hereafter) will be placed (prior art FIG. 1c). Important for the delivery of these stents 12 is to stay in a determined “landing zone”.

The aim is to place the stent graft 12 in a healthy area without occluding any important vessel branches, like e.g. the renal arteries. A sensitive point during the intervention is a release of the main stent graft 12 in the aorta (prior art FIG. 1c). Sometimes the finite stent graft must be mounted from different stent parts, e.g. from stent grafts covering the leg arteries, the aorta etc.

To summarize, the Abdominal Aortic Aneurysm (AAA) disease of the abdominal aorta 10 is shown in FIG. 1a. It is treated either intravascular or via inserting (prior art FIG. 1c) the stent graft 12. Through the groins, the guide wires 11 and catheters are inserted (prior art FIG. 1b) through which the stent graft 12 is inserted (prior art FIG. 1c).

To not have to inject contrast medium permanently to control this complex stent positioning, it is possible to overlay a registered 3D volume 13 showing the segmented relevant part 9 of the aorta 10 to guide the positioning of the stent (prior art—FIG. 2a). Knowing the registration of the volume 13 to a C-arm of a CT scan imaging machine (a C-arm is a rotatable arm of a CT imaging system containing x-ray emitters and detectors—well known in the art) and projection geometry 14, the volume 13 can be projected anatomically correct to a 2D fluoro image 15. This image 15 is shown in a front view in prior art FIG. 2b.

To summarize, if a 3D volume 13 is registered to the C-arm and the projection geometry 14 of the C-arm is known (FIG. 2a), the 3D volume 13 can be overlaid anatomically correct to the 2D fluoroscopic image 15 known as a 2D3D overlay. The visualization can also follow each angle change etc. of the C-arm.

A main problem of the above method is the registration of a CT dataset containing the segmented aorta 10 to the C-arm. Usually this is done using a 3D3D registration method (prior art FIG. 3). To register the segmented aorta 17 to the C-arm (3D3D registration), a 3D volume 16 is acquired on the C-arm (A), which is, via the calibration, implicitly registered to the C-arm. This volume 16 is registered to an external CT(B) using a 3D3D Registration (C). This results in a transformation T shown at (C) which describes the transformation of a CT coordinate system to a coordinate system of the C-arm. If this transformation is applied to the CT volume, it is then also registered to the C-arm (D).

Alternatively, a 2D3D Registration method (prior art FIG. 4) can be used, where usually two angiographies 18, 19 of the aorta 20 have to be taken to register the 3D aorta to the C-arm. Thus FIG. 4 shows possibilities to register a pre-segmented aorta to the C-arm (2D3D Registration using two view projections 18, 19). Here only two projections 18, 19 (preferably 90° apart, e.g. projection 18 lateral and AP projection 19) are acquired on the C-arm (A). To these the external CT volume (B) shown at 8 is registered using 2D3D Registration shown at (C). This results again in a transformation T, which describes the transformation of the CT coordinate system to the C-arm coordinate system. If this transformation is supplied to the CT volume, it is again registered to the C—arm (D).

Thus it is known in the prior art to provide:
3D3D Registration of two volumes;
2D3D Registration of a volume and one or more 2D projections; and
(Semi-) automatic segmentation of the aorta in CT data.

### SUMMARY

It is an object to provide a registration method (along with the corresponding workflow) to ensure a registration where the amount of contrast agent used is minimized.

In a method for visualizing placement of a stent in an aorta of a patient with reduced use of contrast agent, a 3D volume image of the aorta of the patient is provided from a CT scan before placing the stent. An angiography system with a C-arm is provided to take 2D images of the patient. A computer is provided having registration software for registering the 3D volume image and 2D images taken by the angiography system. A first segmentation is performed on the 3D volume image to segment the aorta from remaining parts of the 3D image. A second segmentation is performed using the first segmentation on the 3D volume image to segment a bony structure of the patient from remaining parts of the 3D volume image. A first 2D image of the aorta is obtained from a first direction with use of a contrast agent. A second 2D image is obtained from a second direction but without use of contrast agent. The segmented aorta in the 3D volume image is registered to the C-arm to create a registered 3D volume image by registering the first 2D image to the segmented aorta and registering the second 2D image to the segmented bony structure. The stent is placed in the aorta while observing on the angiography system a third continuous 2D image taken by the angiography system superimposed on the registered 3D volume image.

### BRIEF DESCRIPTION OF THE DRAWINGS

FIG. 1a illustrates an abdominal aortic aneurysm as is known in the prior art;

FIG. 1b shows introduction of a guide wire preliminary to introduction of a stent graft into the segmented aorta to isolate the aneurysm from blood flow as is known in the prior art;

FIG. 1c shows placement of the graft stent to isolate blood flow from the aneurysm as is known in the prior art;

FIG. 2a is a perspective view illustrating a 2D3D overlay technique of the prior art;

FIG. 2b is a frontal view of a 2D fluoro image shown in perspective in FIG. 2a;

FIG. 3 illustrates steps known in the prior art of a 3D3D Registration method;

FIG. 4 shows a 2D3D Registration method according to the prior art;

FIG. 5 illustrates a method according to the preferred embodiment wherein only one angiography is employed along with a native lateral projection to minimize use of contrast agent;

FIG. 6 illustrates preparation for further segmentation of a 3D dataset for automatic registration with different types of images according to the preferred embodiment;

FIG. 7 shows a workflow for a 2D3D registration according to the preferred embodiment using two views with different types of images; and

FIG. 8 is a flow chart of the preferred embodiment method.

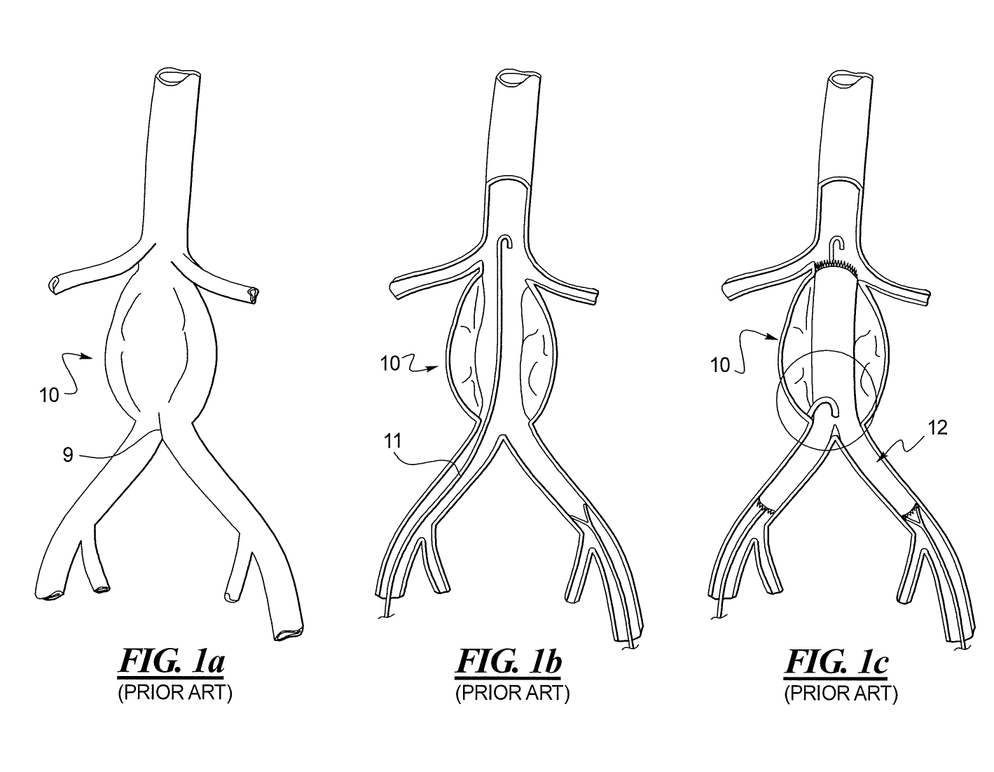

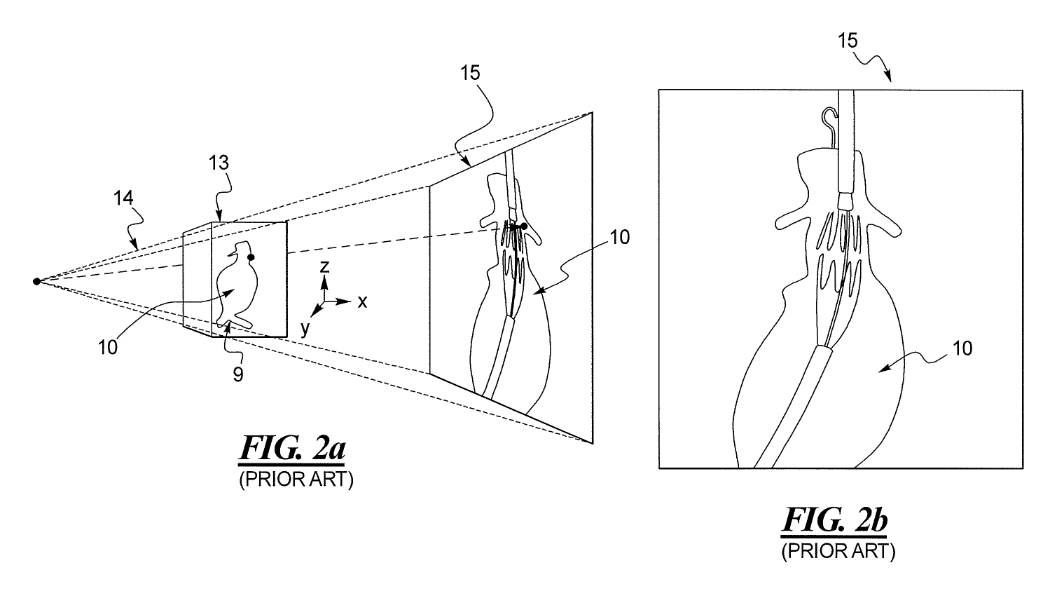

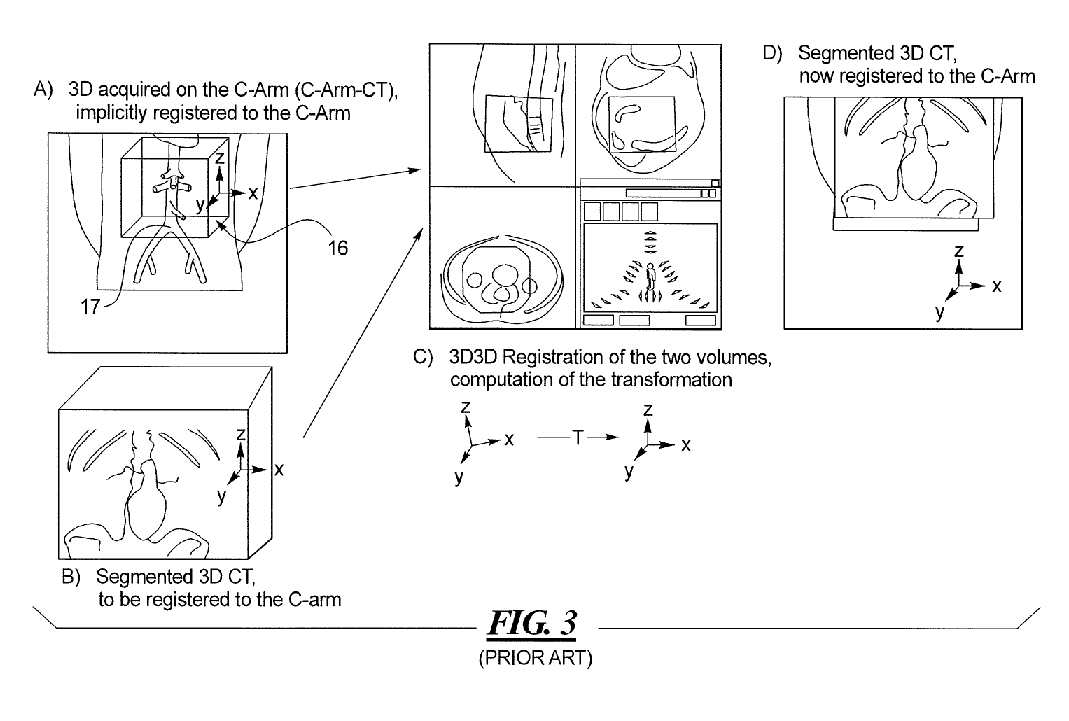

### DESCRIPTION OF THE PREFERRED EMBODIMENT

For the purposes of promoting an understanding of the principles of the invention, reference will now be made to the preferred embodiment/best mode illustrated in the drawings and specific language will be used to describe the same. It will nevertheless be understood that no limitation of the scope of the invention is thereby intended, and such alterations and further modifications in the illustrated device and such further applications of the principles of the invention as illustrated as would normally occur to one skilled in the art to which the invention relates are included.

A registration method of the preferred embodiment with a workflow to register a volume containing a pre-segmented organ is disclosed hereafter. A method is disclosed for registering an abdominal aorta (rsp. aortic aneurysms) to the C-arm but can of course also be extended to any case where pre-segmentation is available, e.g.

thoratic aortas (e.g. for thoratic aneurysms);

aortic roots (e.g. for valve replacement); and

left atria (e.g. for EP ablations).

An object is to ensure a sufficiently good registration without having to apply too much contrast agent.

The method uses a 2D3D Registration from two views (i.e. using two 2D projections) to register the volume to the C-arm. This has the advantage of a much simpler workflow compared to a 3D3D registration approach.

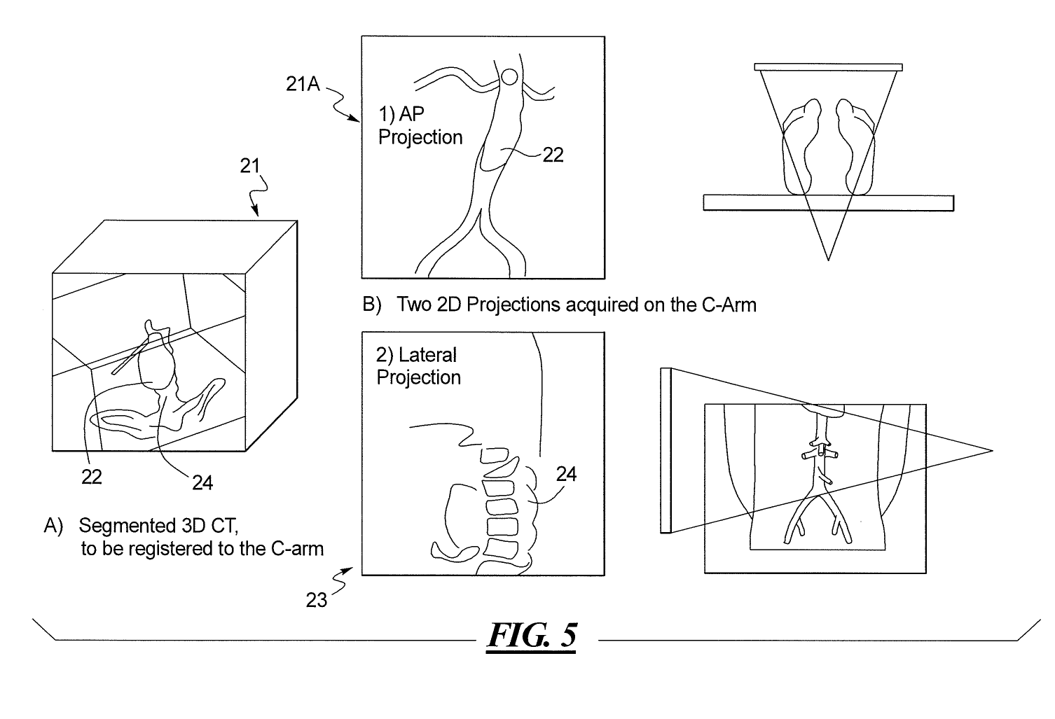

Two 3D angiographies for the registration are not used, but rather as shown in FIG. 5 only one angiography 21 showing the aorta 22 and spine 24 (preferably taken from an AP projection 21A) is employed, along with a native lateral projection 23 showing bony structures (the spine 24).

This gives, on the one hand, accuracy of the registration “3D aorta on 2D aortic angiography”. Also this is an angiography which has to be taken for clinical reasons anyway.

On the other hand, a depth estimation (which is sufficiently accurate using the bony structures, e.g. the spine, as landmarks) can be done without having to apply additional contrast for a second (otherwise unused) angiography.

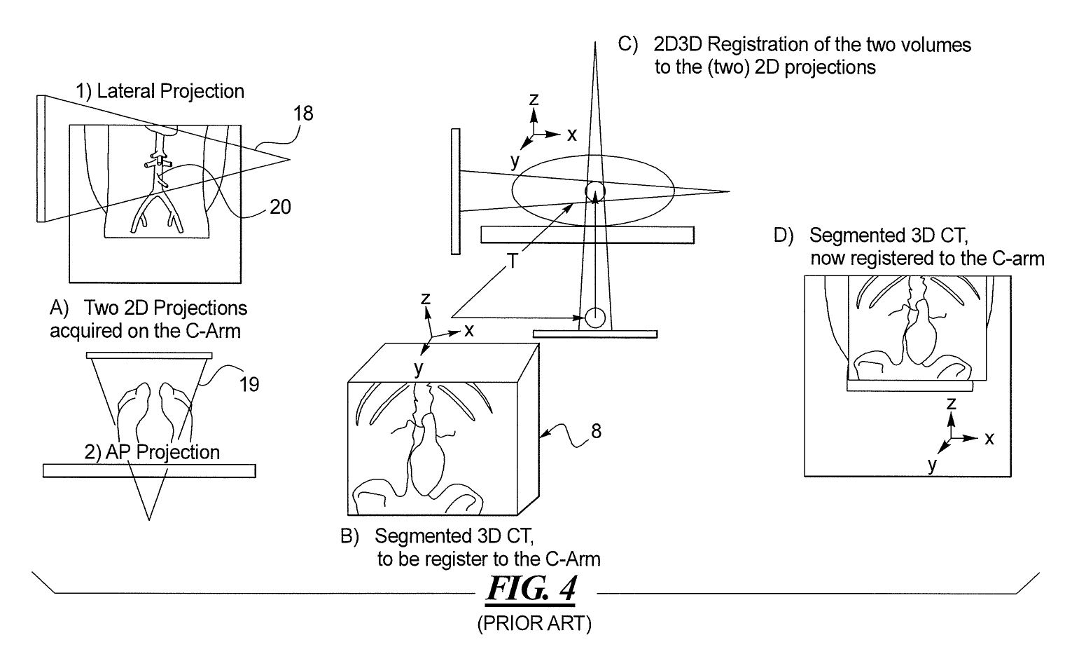

More particularly, FIG. 5 illustrates registering a pre-segmented aorta 24 to the C-arm (2D3D Registration using two views with different types of images). Here the concept is to register the external CT Volume 21 containing the pre-segmented aorta 24, to two 2D projections 21A and 23, as described in FIG. 4. The difference is that for the registration different types of images are used. For the AP projection 21A, an angiography 21 of the aorta 24 is used, which is taken for clinical purpose anyway. The lateral projection 23 is a native image of the spine 24, which gives sufficient depth information, but can be taken without additional contrast agent.

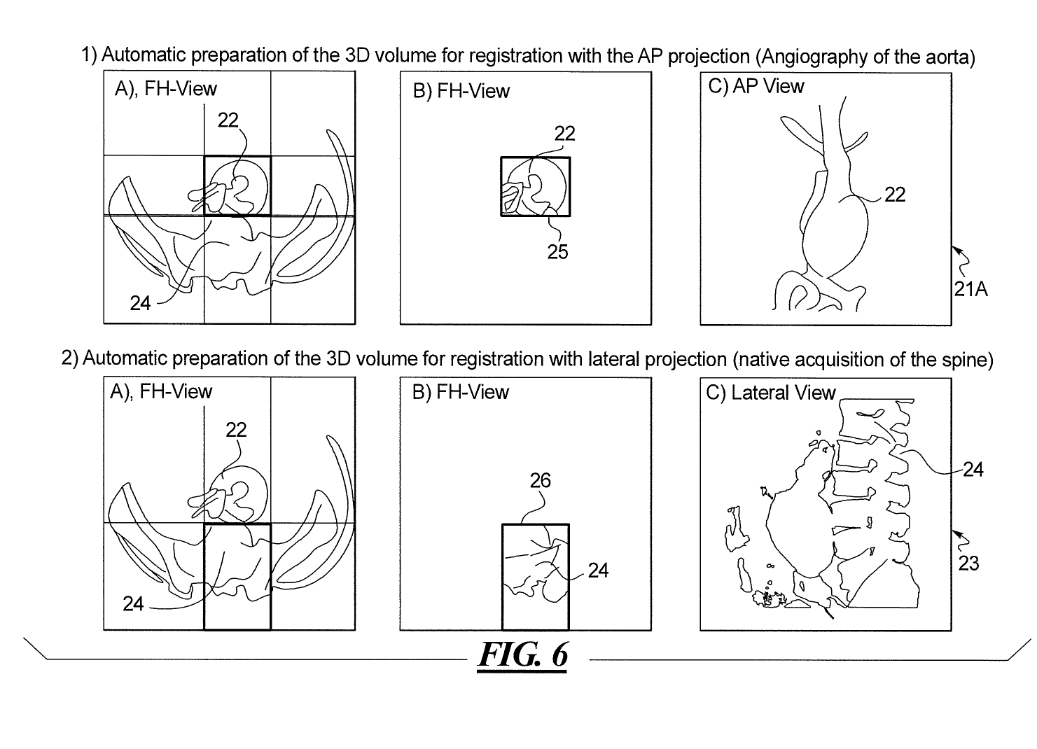

In a further development of the preferred embodiment, as shown in FIG. 6 the 3D volume 21 is further cropped based on the information of the aorta segmentation (or at least knowledge about the position of the aorta in the volume) to be able to optimally register the volume to the different types of 2D images. FIG. 6 shows an example for the AP angiography 21 from AP view 21A and the lateral native acquisition 23 of the spine 24.

For registration of the AP angiography, as shown in FIG. 6 a rectangle 25 is cut out along the known position of the aorta 22, so that the cropped image only contains the aorta 22. This “partial volume” contains only vascular information and is optimally registered with the corresponding angiography.

For registration with the lateral acquisition 23 of the spine 24, a similar approach can be used. Here, another rectangle 26 is cut out described by the width of the aorta 22 (plus a margin so that it definitely contains the spine 24) but below the aorta 22 (FIG. 6). This second “partial volume” then only contains bony information and can be optimally registered with the corresponding native acquisition of the spine 24.

More particularly, FIG. 6 thus shows a preparation (further segmentation) of the 3D dataset for automatic registration with the different types of images. Based on the information about the segmentation of the aorta 22, the 3D CT dataset can further be prepared (i.e. segmented) to better match the different types of 2D projection images used for registration. In FIG. 6, (A) shows a view of the 3D CT data along the aorta 22. For registration of the AP angiography 21 (AP view 21A) the rectangle 25 is cut about along the known position of the aorta 22, so that the cropped image of rectangle 25 only contains the aorta 22 which can optimally be registered with the corresponding angiography.

For registration with the lateral acquisition 23 of the spine 24 the rectangle 26 is cut out described by the width of the aorta 32 (plus a margin) but below it (so that the cropped image of rectangle 26 only contains the spine 24 and can optimally be registered with the corresponding 2D acquisition.

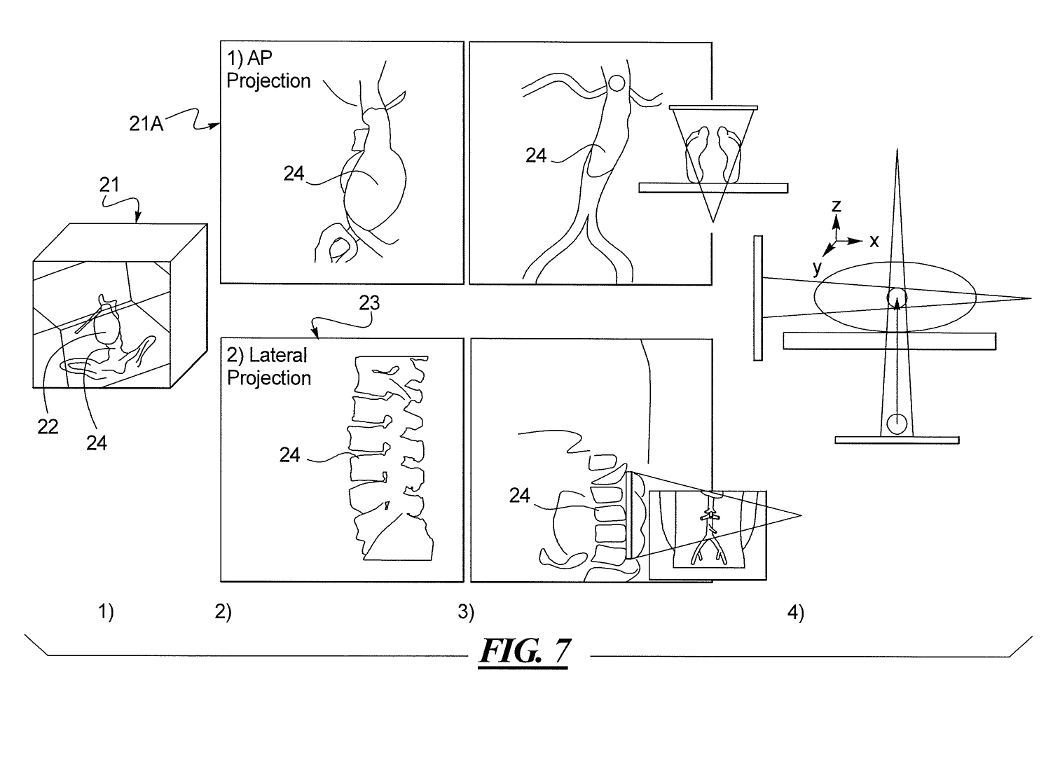

The above gives the following method and workflow for the proposed registration as shown in FIG. 7:

1. pre-segment the aorta 22 in the external angiography CT volume 21 (using a manual, semi-automatic, or automatic step;

2. optionally, the external CT volume 21 is prepared to extract the aorta 22 and the spine 24 for better automatic registration;

3. acquire two 2D projections 21A and 23 (see FIG. 5 also) with the C-arm, preferably 90° apart, e.g.
a. AP projection 21A: Angiography of the aorta 24;
b. Lateral projection 23: Native acquisition of the spine 24; and

4. registration of the two 2D projections 21A and 23 with the pre-segmented (and further prepared) external CT dataset of volume 21, preferably with an automatic (e.g. image based) method.

In particular, FIG. 7 shows the workflow in a 2D3D Registration using two views with different types of images. The workflow contains the following steps:

1) pre-segmentation of the aorta 22 in the external CT volume 21;

2) preparation of the external CT volume 21 to extract the aorta 22 and the spine 24 for later automatic registration;

3) acquisition of two 2D projections 21A and 23 with the C-arm (e.g. AP projection 21A: Angiography 21A/Lateral 23: Native acquisition 23 of the spine 24); and

4) automatic 2D3D registration of the two 2D projections 21A, 23 with the pre-segmented (and further prepared) external CT dataset of the volume 21.

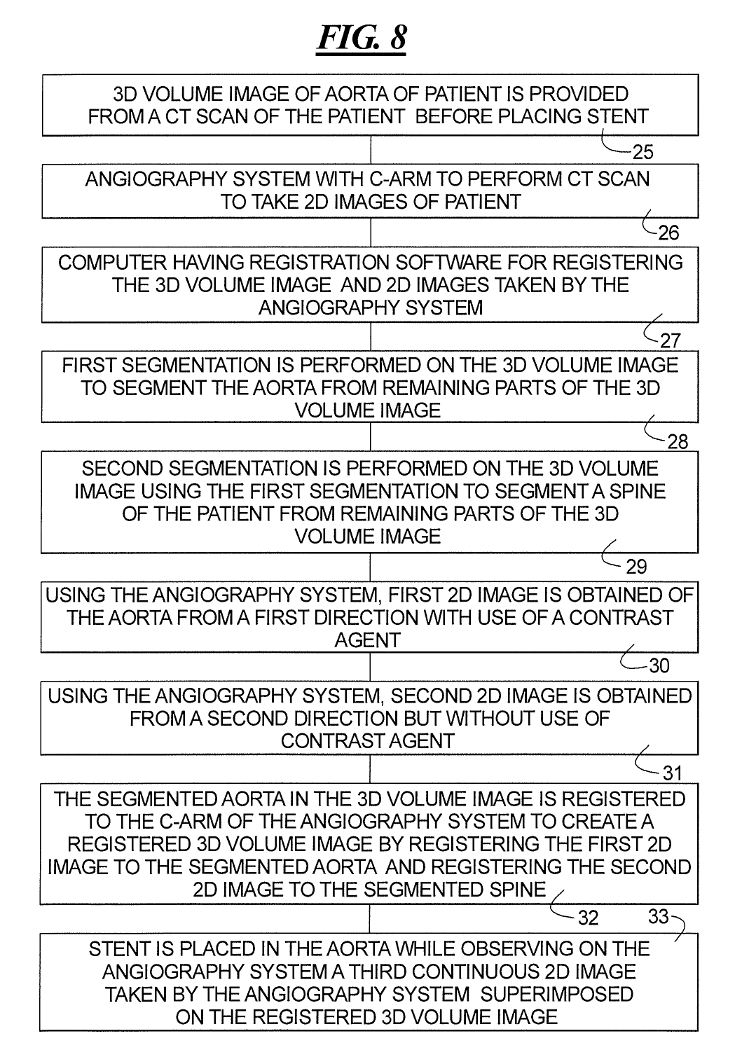

The preferred embodiment method for visualizing placement of the stent in the aorta of the patient with reduced use of contrast agent will now be described with respect to the flow chart shown in FIG. 8.

As shown in block 25, a 3D volume image of the aorta of the patient is provided from a CT scan of the patient before placing the stent.

As shown in block 26, an angiography system with a C-arm is provided, and which can perform a CT scan with the C-arm to take 2D images of the patient.

As shown in block 27, a computer is provided having registration software for registering the 3D volume image and 2D images taken by the angiography system.

As shown in block 28, a first segmentation is performed on the 3D volume image to segment the aorta from remaining parts of the 3D volume image.

As shown in block 29, a second segmentation is performed on the 3D volume image using the first segmentation to segment a spine of the patient from remaining parts of the 3D volume image.

As shown in block 30, using the angiography system, a first 2D image is obtained of the aorta from a first direction with use of a contrast agent.

As shown in block 31, using the angiography system, a second 2D image is obtained from a second direction but without use of contrast agent.

As shown in block 32, the segmented aorta in the 3D volume image is registered to the C-arm of the angiography system to create a registered 3D volume image by registering the first 2D image to the segmented aorta and registering the second 2D image to the segmented spine.

As shown in block 33, the stent is placed in the aorta while observing on the angiography system a third continuous 2D image taken by the angiography system superimposed on the registered 3D volume image.

The method of the preferred embodiment has the following advantages. The proposed reformatting of the 2D3D Registration allows a registration of a CT dataset with a segmented aorta with high accuracy, but with using as minimal a quantity of contrast agent as possible (by using only the clinically indicated angiography along with an uncontrasted image). This improves workflow and patient comfort for a guided procedures using pre-segmented external datasets (e.g. CT volumes).

While the invention has been illustrated and described in detail in the drawings in the above description, the same is to be considered as illustrative and not restrictive in character, it being understood that only the preferred embodiment has been shown and described and that all changes and modifications that come within the scope of the invention are desired to be protected.

I claim as my invention:

1. A method for visualizing placement of a stent in an aorta of a patient with reduced use of contrast agent, comprising the steps of:
providing a 3D volume image of the aorta of the patient from a CT scan of the patient before placing the stent;
providing an angiography system with a C-arm, and which can perform a CT scan with the C-arm to take 2D images of the patient;
providing a computer having registration software for registering the 3D volume image and 2D images taken by the angiography system;
performing a first segmentation on the 3D volume image to segment the aorta from remaining parts of the 3D image;
using said first segmentation to perform a second segmentation on the 3D volume image to segment a bony structure of the patient from remaining parts of the 3D volume image;
using the angiography system obtaining a first 2D image of the aorta from a first direction with use of a contrast agent;
using the angiography system obtaining a second 2D image from a second direction but without use of contrast agent;
registering the segmented aorta in the 3D volume image to the C-arm of the angiography system to create a registered 3D volume image by registering the first 2D image to the segmented aorta and registering the second 2D image to the segmented bony structure; and
placing the stent in the aorta while observing on said angiography system a third continuous 2D image taken by said angiography system superimposed on the registered 3D volume image.

2. The method of claim 1 wherein said second segmentation comprises a rough segmentation.

3. The method of claim 1 wherein the second direction for the second 2D image is at a substantial 90° to the first direction for the first 2D image.

4. The method of claim 1 wherein the aorta comprises an abdominal aorta, and the bony structure comprises the spine.

5. The method of claim 1 wherein the aorta is one of the elements selected from the group consisting of an abdominal aorta, a thoratic aorta, an aortic root, and a left atria.

6. A method for visualizing placement of a stent in an aorta of a patient with reduced use of contrast agent, comprising the steps of:
providing a 3D volume image of the abdominal aorta of the patient from a CT scan of the patient before placing the stent;
providing an angiography system with a C-arm, and which can perform a CT scan with the C-arm to take 2D images of the patient;
providing a computer having registration software for registering the 3D volume image and 2D images taken by the angiography system;
performing a first segmentation on the 3D volume image to segment the aorta from remaining parts of the 3D image;
using said first segmentation to perform a second segmentation on the 3D volume image to segment a spine of the patient from remaining parts of the 3D volume image;
using the angiography system obtaining a first 2D image of the aorta from a first direction with use of a contrast agent;
using the angiography system obtaining a second 2D image from a second direction but without use of contrast agent;
registering the segmented aorta in the 3D volume image to the C-arm of the angiography system to create a registered 3D volume image by registering the first 2D image to the segmented aorta and registering the second 2D image to the segmented spine; and
placing the stent in the aorta while observing on said angiography system a third continuous 2D image taken by said angiography system superimposed on the registered 3D volume image.

7. A method for visualizing placement of a stent in an aorta of a patient with reduced use of contrast agent, comprising the steps of:
providing a 3D volume image of the aorta of the patient from a CT scan of the patient before placing the stent;
providing an angiography system with a C-arm, and which can perform a CT scan with the C-arm to take 2D images of the patient;
providing a computer having registration software for registering the 3D volume image and 2D images taken by the angiography system;
using the angiography system obtaining a first 2D image of the aorta from a first direction with use of a contrast agent;
using the angiography system obtaining a second 2D image from a second direction but without use of contrast agent;
registering the aorta in the 3D volume image to the C-arm of the angiography system to create a registered 3D volume image by registering the first 2D image to the aorta and registering the second 2D image to the bony structure; and
placing the stent in the aorta while observing on said angiography system a third continuous 2D image taken by said angiography system superimposed on the registered 3D volume image.
